(19) United States
(12) Patent Application Publication
(10) Pub. No.: US 2025/0298036 A1
(43) Pub. Date: Sep. 25, 2025

(54) METHODS FOR NONINVASIVE MONITORING OF BRONCHOPULMONARY DYSPLASIA

(71) Applicant: The Children`s Medical Center Corporation, Boston, MA (US)

(72) Inventors: Hanno Steen, Brighton, MA (US); Saima Ahmed, Foxborough, MA (US)

(21) Appl. No.: 18/712,787

(22) PCT Filed: Nov. 22, 2022
(86) PCT No.: PCT/US2022/050788

§ 371 Date: May 23, 2024

Related U.S. Application Data
(60) Provisional application No. 63/282,597, filed on Nov. 23, 2021.

(51) Int. Cl. G01N 33/68; G01N 30/88

(52) U.S. Cl. CPC G01N 33/6893; G01N 2030/8831; G01N 2570/00; G01N 2800/12; G01N 2800/52

(57) ABSTRACT

Provided herein are methods for diagnosing and treating a pulmonary disease, e.g., bronchopulmonary dysplasia (BPD) using proteomics analysis.

### CROSS-REFERENCE TO RELATED APPLICATIONS

This application is the national stage entry of International Patent Application No. PCT/US2022/050788, filed on Nov. 22, 2022, and claims priority to U.S. Provisional Patent Application No. 63/282,597, filed Nov. 23, 2021. The entire content of the foregoing applications is incorporated herein by reference.

### FEDERALLY SPONSORED RESEARCH OR DEVELOPMENT

This invention was made with government support under grant numbers T32HL110852 awarded by the National Institutes of Health (NIH)/National Heart, Lung, and Blood Institute (NHLBI); R01GM112007 awarded by the National Institutes of Health (NIH)/National Institute of General Medical Sciences (NIGMS); U24AI52179 awarded by the National Institutes of Health (NIH)/National Institute of Allergy and Infectious Diseases (NIAID); and R01HL146128, R01HL055454 and R21AI134025 awarded by the National Institutes of Health (NIH). The government has certain rights in the invention.

### TECHNICAL FIELD

This disclosure relates to characterization of biomarkers for the diagnosis and monitoring of bronchopulmonary dysplasia (BPD), and treatment thereof.

### BACKGROUND

Bronchopulmonary dysplasia (BPD) is a multifactorial chronic lung disease of preterm infants exposed to oxygen toxicity and ventilator-induced injury but may also occur in immature infants with few signs of initial lung injury (see, e.g., Bhandari A, Bhandari V. Biomarkers in bronchopulmonary dysplasia. Paediatr Respir Rev. 2013; 14 (3): 173-9). Postnatal mechanical ventilation, prenatal inflammation, infection, maternal preeclampsia, and intrauterine growth restriction are risk factors for BPD. BPD is associated with significant long-term pulmonary morbidities, including airway hyperreactivity and in some cases, emphysematous changes that persist into adulthood (see, e.g., Al-Ghanem G, Shah P, Thomas S, Banfield L, El Helou S, Fusch C, et al. Bronchopulmonary dysplasia and pulmonary hypertension: a meta-analysis. J Perinatol. 2017;37 (4): 414-9). Pulmonary hypertension occurs in approximately 25% of infants with severe BPD and is associated with significant mortality (see, e.g., Al-Ghanem G, Shah P, Thomas S, Banfield L, El Helou S, Fusch C, et al. Bronchopulmonary dysplasia and pulmonary hypertension: a meta-analysis. J Perinatol. 2017;37 (4): 414-9). While case definitions for BPD have evolved over time (see, e.g., Bhandari A, Bhandari V. Biomarkers in bronchopulmonary dysplasia. Paediatr Respir Rev. 2013; 14 (3): 173-9), the reported rate of BPD in the U.S. ranges from 18-89% in extremely preterm infants (<28 weeks gestational age) (see, e.g., Siffel C, Kistler K D, Lewis J FM, Sarda S P. Global incidence of bronchopulmonary dysplasia among extremely preterm infants: a systematic literature review. J Matern Fetal Neonatal Med. 2019:1-11). Thus, BPD remains one of the most common complication of prematurity despite advances in neonatal critical care including the widespread use of non-invasive ventilation.

Current techniques to diagnose and/or monitor critically ill neonates with bronchopulmonary dysplasia (BPD) require invasive sampling of body fluids, which is sub-optimal in these frail neonates. Investigation of validated biomarkers offers the potential of identifying infants with evolving BPD for therapeutic interventions that might eliminate or ameliorate disease. However, fewer infants have tracheal aspirates available because of diminished use of invasive ventilation. Limited blood volume and risk of iatrogenic anemia due to the need for serial measurements, especially in the case of ELGANs, poses a challenge for identifying biomarkers.

Accordingly, there exists a need for a minimally invasive methods for the detection and monitoring of bronchopulmonary dysplasia.

### SUMMARY

Provided herein are methods for non-invasively detecting and monitoring BPD by sampling urine instead of blood. The methods described herein use urine samples for proteomics from extremely low gestational age newborns (ELGANS) at risk for bronchopulmonary dysplasia to characterize proteins and biomarkers associated with BPD.

Accordingly, in one aspect, provided herein are methods of characterizing a urinary protein panel in a subject having a pulmonary disease or disorder, comprising: (a) obtaining a urine sample from the subject; (b) performing a mass spectrometry analysis on a panel of proteins in the urine sample; and (c) identifying one or more proteins that shows significant differences in abundance, as compared to a control sample, thereby characterizing the urinary protein panel.

In one aspect, provided herein are methods of detecting a pulmonary disease or disorder in a subject, comprising (a) obtaining a urine sample from the subject; (b) detecting the abundance of one or more proteins selected from the group consisting of CHI3L1 (UniProt ID P36222), MMP9 (UniProt ID P14780), HSPA8 (UniProt ID P111420, C2 (UniProt ID P06681), FZD6 (UniProt ID 060353), HSPA7 (UniProt ID P48741), ACTB (UniProt ID P60709), FKBPIA (UniProt ID P62942), RPS23 (UniProt ID P62266), CAPNI (UniProt ID P07384), PSMAI (UniProt ID P25786), LRRC4B (UniProt ID Q9NT99), TUBA3C (UniProt ID PODPH7), PKM (UniProt ID P14618), C1QC (UniProt ID P02747), CP (UniProt ID P00450), LCP1 (UniProt ID P13796), CAPZB (UniProt ID P47756), ARHGDIB (UniProt ID P52566), and RAB11A (UniProt ID P62491) in the urine sample; and (c) identifying the subject as having the pulmonary disease or disorder if the one or more proteins have significant differences in abundance, as compared to a control sample.

In some embodiments, the subject is an extremely low gestational age newborn (ELGAN).

In some embodiments, the one or more proteins are chitinase-3-like protein-1 (CHI3L1, also known as YKL-40) and frizzled-6 (FZD6).

In some embodiments, the one or more proteins are matrix metalloproteinase-9 (MMP-9), Tubulin Alpha 3C (TUBA3C), and F-actin-capping protein subunit beta (CAPZB). In some embodiments, the pulmonary disease is bronchopulmonary dysplasia (BPD).

In some embodiments, the control sample is a urine sample from a healthy subject.

In some embodiments, the healthy subject is an age-matched subject.

In some embodiments, the abundance of the one or more proteins are upregulated in a subject having BPD.

In some embodiments, the one or more proteins are selected from the group consisting of CHI3L1 (UniProt ID P36222), MMP9 (UniProt ID P14780), C2 (UniProt ID P06681), FZD6 (UniProt ID 060353), FKBP1A (UniProt ID P62942), LRRC4B (UniProt ID Q9NT99), C1QC (UniProt ID P02747), CP (UniProt ID P00450), LCP1 (UniProt ID P13796), ARHGDIB (UniProt ID P52566), and RAB11A (UniProt ID P62491).

In some embodiments, the methods described herein further comprise determining that the abundance of one or more proteins is downregulated.

In some embodiments, the one or more proteins are selected from the group consisting of HSPA8 (UniProt ID P111420), HSPA7 (UniProt ID P48741), ACTB (UniProt ID P60709), RPS23 (UniProt ID P62266), CAPNI (UniProt ID P07384), TUBA3C (UniProt ID PODPH7), PKM (UniProt ID P14618), and CAPZB (UniProt ID P47756).

In one aspect, provided herein are methods of diagnosing bronchopulmonary dysplasia (BPD) in a subject, comprising: (a) obtaining a urine sample from the subject; (b) detecting the abundance of one or more proteins selected from the group consisting of CHI3L1 (UniProt ID P36222), MMP9 (UniProt ID P14780), HSPA8 (UniProt ID P111420, C2 (UniProt ID P06681), FZD6 (UniProt ID 060353), HSPA7 (UniProt ID P48741), ACTB (UniProt ID P60709), FKBPIA (UniProt ID P62942), RPS23 (UniProt ID P62266), CAPNI (UniProt ID P07384), PSMA1 (UniProt ID P25786), LRRC4B (UniProt ID Q9NT99), TUBA3C (UniProt ID PODPH7), PKM (UniProt ID P14618), C1QC (UniProt ID P02747), CP (UniProt ID P00450), LCP1 (UniProt ID P13796), CAPZB (UniProt ID P47756), ARHGDIB (UniProt ID P52566), and RAB11A (UniProt ID P62491) in the urine sample; and (c) identifying the subject as bronchopulmonary dysplasia (BPD) if the one or more proteins show significant differences in abundance, as compared to a control sample.

In some embodiments, the control sample is a urine sample from a healthy subject.

In some embodiments, the healthy subject is an age-matched subject.

In some embodiments, the abundance of the one or more proteins is upregulated in a subject having BPD. In some embodiments, the one or more proteins are selected from the group consisting of CHI3L1 (UniProt ID P36222), MMP9 (UniProt ID P14780), C2 (UniProt ID P06681), FZD6 (UniProt ID 060353), FKBP1A (UniProt ID P62942), LRRC4B (UniProt ID Q9NT99), C1QC (UniProt ID P02747), CP (UniProt ID P00450), LCP1 (UniProt ID P13796), ARHGDIB (UniProt ID P52566), and RAB11A (UniProt ID P62491).

In some embodiments, the methods described herein further comprise determining that the abundance of one or more proteins is downregulated. In some embodiments, wherein the one or more proteins are selected from the group consisting of HSPA8 (UniProt ID P111420), HSPA7 (UniProt ID P48741), ACTB (UniProt ID P60709), RPS23 (UniProt ID P62266), CAPNI (UniProt ID P07384), TUBA3C (UniProt ID PODPH7), PKM (UniProt ID P14618), and CAPZB (UniProt ID P47756).

In some embodiments, the one or more proteins are chitinase-3-like protein-1 (CHI3L1, also known as YKL-40) and frizzled-6 (FZD6).

In some embodiments, the one or more proteins are selected from the group consisting of matrix metalloproteinase-9 (MMP-9), Tubulin Alpha 3C (TUBA3C), and F-actin-capping protein subunit beta (CAPZB).

In one aspect, provided herein are methods of predicting a response to a drug for bronchopulmonary dysplasia (BPD) in a subject, comprising: (a) obtaining a urine sample from the subject; (b) detecting the abundance of one or more proteins selected from Table 2 in the urine sample; and (c) identifying the subject as potential responder for the BPD drug if the one or more proteins show significant differences in abundance, compared to a control sample.

In some embodiments, the BPD drug is selected from a small molecule drug, an antibody or antigen-binding fragment thereof, an oligonucleotide or a combination thereof.

In some embodiments, the BPD drug is selected from Marimastat, Captopril, Tacrolimus, Pimecrolimus, Bendroflumethiazide, Celecoxib, Miglitol, Bumetanide, Artenimol, Stiripentol, Artenimol, Dasatinib, Halofuginone, Dalteparin, Artenimol, Cholecystokinin, Oxibendazole, Dasatinib, and Diflucorolone.

In some embodiments, the control sample is a urine sample from a healthy subject.

In some embodiments, the healthy subject is an age-matched subject.

In some embodiments, the subject is an extremely low gestational age newborn (ELGAN).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic illustration of the in-house MStern blotting-based urine proteomics platform including Liquid Chromatography/Mass Spectrometry acquisition and data analysis which was used to process as little as 50 μL of ELGANs premature infant urine collected within 72 hours of birth.

FIGS. 2A-2B show that the urine proteome identifies significantly different proteins infants with BPD versus healthy controls. FIG. 2A is a volcano plot analysis of 21 cases (right side) and 21 controls (left side) with p value cutoff of 0.05 (equivalent to −log 10 (p)>1.3); and FIG. 2B is a table of top 20 significant proteins as indicated in volcano plot with p value, Log2 fold change, fold change direction, and literature reference. Bolded proteins are described specifically in BPD and or pediatric respiratory pathologies literature. Rows in light grey are proteins significantly upregulated in BPD. Rows in dark grey are proteins significantly upregulated in controls.

FIGS. 3A-3D show that Gene Ontology (GO) analysis of urinary proteomes in BPD identifies proteins in immune effector process and neutrophil degranulation. Shown are the top 20 GO annotation biological processes based on the FDR score in a STRING analysis for (FIG. 3A) the upregulated proteins (n=35) and (FIG. 3B) the downregulated proteins (n=59). STRING protein-protein network with the proteins linked to the GO annotation immune effector process highlighted (FIG. 3C). STRING protein-protein network with proteins linked to neutrophil degranulation highlighted (FIG. 3D).

FIGS. 4A-4H show the significant proteins previously described in BPD and or pediatric respiratory pathologies literature including two proteins (MMP-9 and FKBP1A) which are FDA approved drug targets. Box plot of control vs BPD with p value and ROC with AUROC and 95% confidence interval are shown for each protein CHI3L1 (FIG. 4A), MMP-9 (FIG. 4B), CP (FIG. 4C), FZD6 (FIG. 4D), C1QC (FIG. 4E), C2 (FIG. 4F), and FKBPIA (FIG. 4G). FIG. 4H is an ROC graph of the preliminary biomarker panel including the two biomarker candidates that make up the panel. For each the AUROC are given.

FIGS. 5A-5C show the logistical regression analysis for gestational age (FIG. 5A), birth weight (FIG. 5B), and sex (FIG. 5C) included in BPD urine proteomics analysis.

FIGS. 6A-6B shows a 3-protein biomarker panel identified in urine proteomics analysis includes previously validated (MMP-9) and novel (TUBA3C and CAPZB) BPD-associated proteins. FIG. 6A is a box plot of control vs BPD with p value and ROC with AUROC and 95% confidence interval are shown for 3-protein biomarker panel. FIG. 6B is an ROC graph of the preliminary biomarker panel including the three biomarker candidates that make up the panel. For each the AUROC are given.

FIG. 7 is a table showing co-morbidities of Entire Parent Cohort from which BPD urine proteomics analysis samples were selected.

FIG. 8 is a table showing the race and ethnicity of patients used in BPD urine proteomics analysis.

### DETAILED DESCRIPTION

Provided herein are methods for characterizing a urinary protein panel in a subject having a pulmonary disease or disorder (e.g., BPD). Also provided herein are methods for detecting a pulmonary disease or disorder (e.g., BPD) in a subject. Also provided herein are methods of diagnosing BPD or predicting a response to a drug for BPD in a subject.

The current disclosure is based on, at least partially, on the discovery that urine proteomics can be used to identify therapeutic target candidates and/or might serve as indicators of the disease course or response to treatment of a particular preterm neonate.

### Bronchopulmonary Dysplasia (BPD)

Bronchopulmonary dysplasia (BPD) is a form of chronic lung disease that affects newborns. Most infants who develop BPD have been born prematurely and need oxygen therapy. Most infants recover from BPD, but some may have long-term breathing difficulties.

The clinical diagnosis of BPD is made in any prematurely born infant who, at 36 weeks gestation, has lung disease requiring continuous or continual supplemental oxygen and who has had an abnormal chest X-ray. As used herein, “BPD” also includes all alternative clinical diagnosis definitions, such as a diagnosis in infants older than four weeks from birth who have had persistent lung disease requiring continual supplemental oxygen and who have had abnormal chest X-rays. BPD is also sometimes referred to in the literature and by pediatric caretakers as “chronic lung disease” (see, e.g., Jobe et al., Early Hum. Devel. 53:81-94 (1998)).

Because BPD clinically is not diagnosed in prematurely born infants until some time after birth, e.g. 36 weeks gestation, therapies to treat at-risk infants are typically administered before the disease is formally diagnosed. In some methods, therapies are used to treat infants who have been diagnosed with BPD to lessen the severity of the disease.

BPD occurs in very ill infants who received high levels of oxygen for a long period. BPD can also occur in infants who were on a breathing machine (ventilator). BPD is more common in infants born early (prematurely), whose lungs were not fully developed at birth.

The risk factors for BPD include, for example, congenital heart disease (problem with the heart's structure and function that is present at birth), prematurity, usually in infants born before 32 weeks gestation, and severe respiratory or lung infection

The symptoms of BPD include, for example, bluish skin color (cyanosis), cough, rapid breathing, and shortness of breath

Exams and tests that may be done to help diagnose BPD include, for example, arterial blood gas, chest CT scan, chest X-ray, or pulse oximetry.

### Methods of Characterizing Urinary Protein Panels and Detecting Pulmonary Diseases

In one aspect, provided herein are methods of characterizing a urinary protein panel in a subject having a pulmonary disease or disorder, comprising: (a) obtaining a urine sample from the subject; (b) performing an analysis (e.g., mass spectrometry) on a panel of a proteins in the urine sample; (c) identifying one or more proteins that show significant differences in abundance, compared to a control sample, thereby characterizing the urinary protein panel.

In another aspect, provided herein are methods of detecting a pulmonary disease or disorder in a subject, comprising (a) obtaining a urine sample from the subject; (b) detecting the abundance of one or more proteins selected from the group of CHI3L1 (UniProt ID P36222), MMP9 (UniProt ID P14780), HSPA8 (UniProt ID P111420), C2 (UniProt ID P06681), FZD6 (UniProt ID 060353), HSPA7 (UniProt ID P48741), ACTB (UniProt ID P60709), FKBPIA (UniProt ID P62942), RPS23 (UniProt ID P62266), CAPNI (UniProt ID P07384), PSMAI (UniProt ID P25786), LRRC4B (UniProt ID Q9NT99), TUBA3C (UniProt ID PODPH7), PKM (UniProt ID P14618), C1QC (UniProt ID P02747), CP (UniProt ID P00450), LCP1 (UniProt ID P13796), CAPZB (UniProt ID P47756), ARHGDIB (UniProt ID P52566), and RAB11A (UniProt ID P62491) in the urine sample; and (c) identifying the subject as having the pulmonary disease if the one or more proteins show significant differences in abundance, compared to a control sample.

In another aspect, provided herein are methods of diagnosing bronchopulmonary dysplasia (BPD) in a subject, comprising: (a) obtaining a urine sample from the subject; (b) detecting the abundance of one or more proteins selected from the group of CHI3L1 (UniProt ID P36222), MMP9 (UniProt ID P14780), HSPA8 (UniProt ID P111420), C2 (UniProt ID P06681), FZD6 (UniProt ID 060353), HSPA7 (UniProt ID P48741), ACTB (UniProt ID P60709), FKBP1A (UniProt ID P62942), RPS23 (UniProt ID P62266), CAPNI (UniProt ID P07384), PSMAI (UniProt ID P25786), LRRC4B (UniProt ID Q9NT99), TUBA3C (UniProt ID PODPH7), PKM (UniProt ID P14618), CIQC (UniProt ID P02747), CP (UniProt ID P00450), LCP1 (UniProt ID P13796), CAPZB (UniProt ID P47756), ARHGDIB (UniProt ID P52566), and RAB11A (UniProt ID P62491) in the urine sample; (c) identifying the subject as bronchopulmonary dysplasia (BPD) if the one or more proteins show significant differences in abundance, compared to a control sample.

In some embodiments, the pulmonary disease is bronchopulmonary dysplasia (BPD).

In some embodiments, the methods described herein assessing the expression level of at least 1, 2, 3, 4, 5, 6, 7, 8, 9, 10, 11, 12, 13, 14, 15, 16, 17, 18, or 19 protein(s) selected from CHI3L1 (UniProt ID P36222), MMP9 (UniProt ID P14780), HSPA8 (UniProt ID P111420), C2 (UniProt ID P06681), FZD6 (UniProt ID 060353), HSPA7 (UniProt ID P48741), ACTB (UniProt ID P60709), FKBPIA (UniProt ID P62942), RPS23 (UniProt ID P62266), CAPNI (UniProt ID P07384), PSMA1 (UniProt ID P25786), LRRC4B (UniProt ID Q9NT99), TUBA3C (UniProt ID PODPH7), PKM (UniProt ID P14618), CIQC (UniProt ID P02747), CP (UniProt ID P00450), LCP1 (UniProt ID P13796), CAPZB (UniProt ID P47756), ARHGDIB (UniProt ID P52566), and RAB11A (UniProt ID P62491).

In some embodiments, the methods described herein include identifying that one or more proteins are upregulated in a BPD subject compared to a healthy subject. In some embodiments, the methods described herein include identifying that one or more proteins are downregulated in a BPD subject compared to a healthy subject. In some embodiments, methods described herein include identifying that one or more proteins are upregulated in a healthy subject. In some embodiments, the methods described herein include identifying that one or more proteins are upregulated in a BPD subject and not in a healthy subject. In some embodiments, the methods described herein include identifying that one or more proteins are downregulated in a BPD subject and not in a healthy subject. In some embodiments, the methods described herein include identifying that one or more proteins are upregulated in a healthy subject and not in a BPD subject.

In some embodiments, the one or more proteins that are upregulated in a subject having BPD include one or more of CHI3L1 (UniProt ID P36222), MMP9 (UniProt ID P14780), C2 (UniProt ID P06681), FZD6 (UniProt ID 060353), FKBPIA (UniProt ID P62942), LRRC4B (UniProt ID Q9NT99), CIQC (UniProt ID P02747), CP (UniProt ID P00450), LCP1 (UniProt ID P13796), ARHGDIB (UniProt ID P52566), and RAB11A (UniProt ID P62491). In some embodiments, the one or more proteins are upregulated in a subject having BPD compared to the abundance of the one or more proteins in a healthy subject.

In some embodiments, the one or more proteins that are upregulated in a subject having BPD but not in a healthy subject include one or more of CHI3L1 (UniProt ID P36222), MMP9 (UniProt ID P14780), C2 (UniProt ID P06681), FZD6 (UniProt ID O60353), FKBP1A (UniProt ID P62942), LRRC4B (UniProt ID Q9NT99), CIQC (UniProt ID P02747), CP (UniProt ID P00450), LCP1 (UniProt ID P13796), ARHGDIB (UniProt ID P52566), and RAB11A (UniProt ID P62491).

In some embodiments, the one or more proteins that are downregulated in a subject having BPD include one or more of HSPA8 (UniProt ID P111420), HSPA7 (UniProt ID P48741), ACTB (UniProt ID P60709), RPS23 (UniProt ID P62266), CAPNI (UniProt ID P07384), TUBA3C (UniProt ID PODPH7), PKM (UniProt ID P14618), and CAPZB (UniProt ID P47756). In some embodiments, the one or more proteins are downregulated in a subject having BPD compared to the abundance of the one or more proteins in a healthy subject.

In some embodiments, the one or more proteins that are upregulated in a healthy subject include one or more of HSPA8 (UniProt ID P111420), HSPA7 (UniProt ID P48741), ACTB (UniProt ID P60709), RPS23 (UniProt ID P62266), CAPNI (UniProt ID P07384), TUBA3C (UniProt ID PODPH7), PKM (UniProt ID P14618), and CAPZB (UniProt ID P47756).

In some embodiments, the one or more proteins that are upregulated in a healthy subject but not in a subject having BPD include one or more of HSPA8 (UniProt ID P111420), HSPA7 (UniProt ID P48741), ACTB (UniProt ID P60709), RPS23 (UniProt ID P62266), CAPNI (UniProt ID P07384), TUBA3C (UniProt ID PODPH7), PKM (UniProt ID P14618), and CAPZB (UniProt ID P47756).

In some embodiments, the upregulation of the one or more proteins indicates that there is a higher likelihood that the subject has BPD. In some embodiments, the upregulation of the one or more proteins indicates that the subject has BPD. In some embodiments, the downregulation of the one or more proteins indicates that there is a higher likelihood that the subject has BPD. In some embodiments, the downregulation of the one or more proteins indicates that the subject has BPD. In some embodiments, the upregulation of one or more proteins and the downregulation of other one or more proteins indicates that there is a higher likelihood that the subject has BPD. In some embodiments, the upregulation of one or more proteins and the downregulation of other one or more proteins indicates that the subject has BPD.

In some embodiments, the one or more proteins are chitinase-3-like protein-1 (CHI3L1, also known as YKL-40) and frizzled-6 (FZD6).

In some embodiments, the methods described herein include identifying that CHI3L1 is upregulated in a BPD subject compared to a healthy subject.

In some embodiments, the methods described herein include identifying that FZD6 is upregulated in a BPD subject compared to a healthy subject.

In some embodiments, the methods described herein include identifying that CHI3L1 and FZD6 are upregulated in a BPD subject compared to a healthy subject.

In some embodiments, the one or more proteins are matrix metalloproteinase-9 (MMP-9), Tubulin Alpha 3C (TUBA3C), and F-actin-capping protein subunit beta (CAPZB).

In some embodiments, the methods described herein include identifying that MMP-9 is upregulated in a BPD subject compared to a healthy subject.

In some embodiments, the methods described herein include identifying that TUBA3C is downregulated in a BPD subject compared to a healthy subject.

In some embodiments, the methods described herein include identifying that CAPZB is downregulated in a BPD subject compared to a healthy subject.

In some embodiments, the methods described herein include identifying that MMP-9 is upregulated and that TUBA3C is downregulated in a BPD subject compared to a healthy subject. In some embodiments, the methods described herein include identifying that MMP-9 is upregulated and that CAPZB is downregulated in a BPD subject compared to a healthy subject. In some embodiments, the methods described herein include identifying that TUBA3C and CAPZB are downregulated in a BPD subject compared to a healthy subject. In some embodiments, the methods described herein include identifying that MMP-9 is upregulated and that TUBA3C and CAPZB are downregulated in a BPD subject compared to a healthy subject.

In some embodiments, the abundance of the one or more proteins is upregulated by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100% or more.

In some embodiments, the abundance of the one or more proteins is upregulated by about 2 folds, 3 folds, 4 folds, 5 folds, 6 folds, 7 folds, 8 folds, 9 folds, 10 folds, 11 folds, 12 folds, 13 folds, 14 folds, 15 folds, 16 folds, 17 folds, 18 folds, 19 folds, 20 folds, 25 folds, 30 folds, 35 folds, 40 folds, 45 folds, 50 folds or more.

In some embodiments, the abundance of the one or more proteins is downregulated by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100% or more.

In some embodiments, the abundance of the one or more proteins is downregulated by about 2 folds, 3 folds, 4 folds, 5 folds, 6 folds, 7 folds, 8 folds, 9 folds, 10 folds, 11 folds, 12 folds, 13 folds, 14 folds, 15 folds, 16 folds, 17 folds, 18 folds, 19 folds, 20 folds, 25 folds, 30 folds, 35 folds, 40 folds, 45 folds, 50 folds or more.

Samples for use in the methods described herein include various types of samples from a subject.

In some embodiments, the sample is a “biologic sample”. As used herein, the term “biological sample” or “sample” refers to a sample obtained or derived from a subject. In some embodiments, the sample is a bodily fluid. In some embodiments, the sample is a urine sample.

As used herein, “obtain” or “obtaining” can be any means whereby one comes into possession of the sample by “direct” or “indirect” means. Directly obtaining a sample means performing a process (e.g., performing a physical method such as extraction) to obtain the sample. Indirectly obtaining a sample refers to receiving the sample from another party or source (e.g., a third party laboratory that directly acquired the sample). Directly obtaining a sample includes performing a process that includes a physical change in a physical substance, e.g., a starting material, such as urine. Thus, obtain is used to mean collection and/or removal of the sample from the subject. Furthermore, “obtain” is also used to mean where one receives the sample from another who was in possession of the sample previously.

In some embodiments, a reference sample (control sample) is obtained from at least one individual not suffering from BPD. In some other embodiments, the reference sample is obtained from at least one individual previously diagnosed as having BPD. In some embodiments, the reference sample comprises a predetermined, statistically significant reference analyte levels. In some embodiments, the reference sample is obtained from an age-matched subject. In some embodiments, the reference sample is obtained from a sex-matched subject. In some embodiments, the sample is from a live subject. In some embodiments, the control sample is a urine sample from a healthy subject.

In some embodiments, the methods described herein further includes administering a BPD treatment (e.g., a drug) to the subject. The terms “subject” and “patient” are used interchangeably throughout the specification and describe an animal, human or non-human, to whom treatment according to the methods of the present invention is provided. In some embodiments, the term “subject”, as used herein, refers to a human (e.g., a man, a woman, or a child).

In some embodiments, the subject is a human subject. In some embodiments, the subject is an extremely low gestational age newborn (ELGAN). As used herein, ELGAN refers to a subject born more than 3 months before the expected date of birth.

In some embodiments, the subject is about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks or more in gestational age (time elapsed between the first day of the last menstrual period and the day of delivery).

In some embodiments, the subject is about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks or more in chronological age (time elapsed from birth).

In some embodiments, the subject is about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 53 weeks, about 54 weeks, about 55 weeks, about 56 weeks, about 57 weeks, about 58 weeks, about 59 weeks, about 60 weeks or more in postmenstrual age (gestational age plus chronological age).

In some embodiments, the detection of the pulmonary disease (e.g., BPD) further includes using one or more additional methods or indexes, e.g., arterial blood gas, chest CT scan, chest X-ray, or pulse oximetry for the detection of the disease

### Methods of Treating a BPD Subject or Predicting a Drug Response

In one aspect, provided herein are methods of predicting a response to a drug for bronchopulmonary dysplasia (BPD) in a subject, comprising: (a) obtaining a urine sample from the subject; (b) detecting the abundance of one or more proteins selected from Table 2 in the urine sample; (c) identifying the subject as potential responder for the BPD drug if the one or more proteins show significant differences in abundance, compared to a control sample.

In one aspect, provided herein are methods of treating bronchopulmonary dysplasia (BPD) in a subject, comprising: (a) obtaining a urine sample from the subject; (b) detecting the abundance of one or more proteins selected from Table 2 in the urine sample; (c) identifying the subject as having BPD if the one or more proteins show significant differences in abundance, compared to a control sample; and (d) administering a BPD drug to the subject.

In some embodiments, the methods described herein further comprises monitoring the progression of bronchopulmonary dysplasia (BPD) in a subject, including detecting the abundance of the one or more proteins at a second time point and compare the abundance of the one or more proteins in the first time point, thereby monitoring the progression of BPD.

In some embodiments, the second time point is a time point after one or more treatment of BPD, e.g., the administration of a BPD drug.

In some embodiments, the progression of BPD is monitored over a time period of about 1 week, about 2 weeks, about 3 weeks, about 4 weeks, about 5 weeks or more.

In some embodiments, the progression of BPD is monitored over a time period of about 2 months, about 3 months, about 4 months, about 5 months, about 6 months, about 7 months, about 8 months, about 9 months, about 10 months, about 11 months, about 12 months or more.

In some embodiments, the BPD drug is selected from a small molecule drug, an antibody or antigen-binding fragment thereof, an oligonucleotide or a combination thereof.

In some embodiment, the BPD drug is selected from Marimastat, Captopril, Tacrolimus, Pimecrolimus, Bendroflumethiazide, Celecoxib, Miglitol, Bumetanide, Artenimol, Stiripentol, Artenimol, Dasatinib, Halofuginone, Dalteparin, Artenimol, Cholecystokinin, Oxibendazole, Dasatinib, and Diflucorolone.

In some embodiments, the methods described herein further include treating the subject in combination with other treatment methods.

Examples of types of drug therapies that may be used herein include: (1) Diuretics, which is a class of drugs that helps to decrease the amount of fluid in and around the alveoli. They are usually given by mouth one to four times per day. (2) Bronchodilators, which are medications that help relax the muscles around the air passages, which makes breathing easier by widening the airway openings. They are usually given as an aerosol by a mask over the infant's face and using a nebulizer or an inhaler with a spacer. (3) Corticosteroids, which are drugs that reduce and/or prevent inflammation within the lungs. They help reduce swelling in the windpipe and decrease the amount of mucus that is produced. Like bronchodilators, they are also usually given as an aerosol with a mask, either with the use of a nebulizer or an inhaler with a spacer. (4) Viral immunization: Children with BPD are at increased risk for respiratory tract infections especially respiratory syncytial virus (RSV). Infants with moderate or severe BPD receive monthly injections with a medication that helps prevent the infection during the RSV season. (5) Cardiac medications: A few infants with BPD may require special medications that help relax the muscles around the blood vessels in the lung, allowing the blood to pass more freely and reduce the strain on the heart.

In some embodiments, subjects with more severe disease need oxygen for several months. They may also need some form of support with a machine that delivers pressure through the nose through special prongs or a mask. These machines provide either nasal continuous positive airway pressure (NCPAP) or bilevel positive airway pressure (BiPAP). In some embodiments, subjects with very severe disease need to stay on a ventilator for a long time, in which case they will need to receive a tracheostomy (a breathing tube inserted into the lungs through the neck).

In some embodiments, the BPD drug is an inhibitor (which decreases the expression/activity) of one or more of CHI3L1 (UniProt ID P36222), MMP9 (UniProt ID P14780), C2 (UniProt ID P06681), FZD6 (UniProt ID 060353), FKBP1A (UniProt ID P62942), LRRC4B (UniProt ID Q9NT99), CIQC (UniProt ID P02747), CP (UniProt ID P00450), LCP1 (UniProt ID P13796), ARHGDIB (UniProt ID P52566), and RAB11A (UniProt ID P62491).

In some embodiments, the BPD drug is an activator (which increases the expression/activity) of one or more of HSPA8 (UniProt ID P111420), HSPA7 (UniProt ID P48741), ACTB (UniProt ID P60709), RPS23 (UniProt ID P62266), CAPNI (UniProt ID P07384), TUBA3C (UniProt ID PODPH7), PKM (UniProt ID P14618), and CAPZB (UniProt ID P47756).

In some embodiments, the BPD drug is an inhibitor (which decreases the expression/activity) of one or more of LCP1 (UniProt ID P13796), ARHGDIB (UniProt ID P52566), and RAB11A (UniProt ID P62491).

In some embodiments, the BPD drug is an activator (which increases the expression/activity) of one or more of HSPA8 (UniProt ID P111420), HSPA7 (UniProt ID P48741), ACTB (UniProt ID P60709), RPS23 (UniProt ID P62266), CAPNI (UniProt ID P07384), TUBA3C (UniProt ID PODPH7), PKM (UniProt ID P14618), and CAPZB (UniProt ID P47756).

### Administration of the Agents

In some embodiments, the methods include administering agents, e.g., inhibitors of the one or more proteins identified using the methods described herein, to the subject. Any suitable administration methods known in the art can be used in the methods described herein.

In some embodiments, the methods described herein include the use of pharmaceutical compositions comprising the agent for the inhibition of the expression and/or activity of the one or more proteins identified using the methods described herein.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art (see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active composition in the required amount in an appropriate solvent with one or a combination of ingredients enumerated above, as required, followed by filtered sterilization. Generally, dispersions are prepared by incorporating the active composition into a sterile vehicle, which contains a basic dispersion medium and the required other ingredients from those enumerated above.

The pharmaceutical compositions can be included in a container, pack, or dispenser together with instructions for administration.

### Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of an active agent (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### EXAMPLES

The invention is further described in the following examples, which do not limit the scope of the invention described in the claims.

### Example 1: Urine Proteomics for Noninvasive Monitoring of Biomarkers in Bronchopulmonary Dysplasia

The aim of this study was to demonstrate the benefit of using non-invasively collected microliter amounts of urine for monitoring the health status of ELGANs. We applied a urine proteomics platform to samples collected from ELGANs within 72 hours of birth to confirm proteins previously identified in BPD-focused studies using invasively collected body fluids such as blood, tracheal aspirates, and broncho-alveolar lavage. Furthermore, we aimed to identify additional proteins providing novel insights into the pathophysiology of BPD and potentially serving as novel targets for therapies.

Current techniques to diagnose and/or monitor critically ill neonates with bronchopulmonary dysplasia (BPD) require invasive sampling of body fluids, which is sub-optimal in these frail neonates. Our study does show that disease-associated changes in easily and non-invasively collected urine closely resemble the molecular changes that have been described for blood, tracheal aspirate, and broncho-alveolar lavage. This is advantageous as it highlights our biomarkers to be used as a non-invasive monitoring platform for BPD-associated proteins that can be used for investigating BPD pathogenesis, morbidity, and mortality.

We are proposing new biomarker panels to be used in addition to any other diagnostic modality currently used in the NICU all around the country/world.

We developed a robust high throughput urine proteomics methodology that requires only 50 microliters of urine. We utilized the methodology with a proof-of-concept study validating proteins previously identified in invasive samples such as blood and/or tracheal aspirates on urine collected within 72 hours of birth from ELGANS (gestational age (26+1.2) weeks) who were admitted to a single Neonatal Intensive Care Unit (NICU); half of whom eventually developed BPD (n=21), while the other half served as controls (n=21).

Our high throughput urine proteomics approach clearly identified several BPD-associated changes in the urine proteome recapitulating expected blood proteome changes and several urinary proteins predicted BPD risk. Interestingly, sixteen of the identified urinary proteins are known targets of drugs approved by the Food and Drug Administration.

In addition to validating numerous proteins, previously found in invasively collected blood, tracheal aspirate, and broncho-alveolar lavage, that have been implicated in BPD pathophysiology, urine proteomics also suggested novel potential therapeutic targets. Ease of access to urine could allow for sequential proteomic evaluations for longitudinal monitoring of disease progression and impact of therapeutic intervention in future studies.

### Materials and Methods

### Study Population and Urine Sample Collection

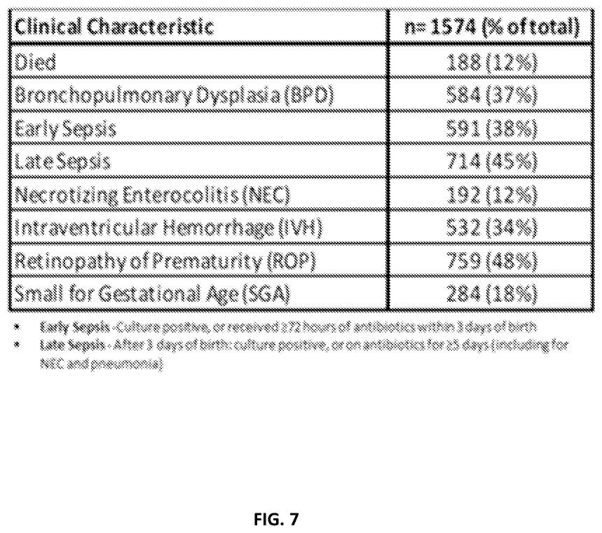

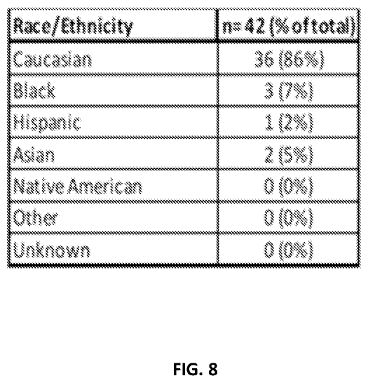

All urine samples were obtained from ELGANs admitted to the Neonatal Intensive Care Unit (NICU) at Brigham and Women's Hospital (BWH), Boston, Massachusetts, between 1997 and 2015 through a Partners Human Research Committee-approved discarded specimen and medical records protocol. All infants in the BWH cohort were born before 29 weeks of gestation. Comorbidities are described in FIG. 7. BPD cases were defined as infants requiring supplemental oxygen at 36 weeks' postmenstrual age (PMA). BPD cases (n=21) were compared with controls from the remaining cohort who did not meet this definition of BPD (n=21). Race/Ethnicity of the 42 samples are described in FIG. 8. Urine was collected within 72 hours of birth using wet cotton balls, processed to remove debris, and stored at −80° C. until use.

### Sample Preparation

MStern Blotting: The widely accepted “MStern Blotting” approach was used to process all urine samples (see, e.g., Bhandari A, Bhandari V. Biomarkers in bronchopulmonary dysplasia. Paediatr Respir Rev. 2013; 14 (3): 173-9) using our in-house robust high throughput proteomics pipeline. In short, to 50-150 μL urine we added dithiothreitol (DTT) and (1:1 (w/w) urea in1 M Tris/HCl pH 8.5 to a final concentration of 10 mM DTT. The resulting denatured and reduced urine was incubated for 20 min at 27° C. and 1100 rpm in a ThermoMixer (Eppendorf). Reduced cysteine side chains were alkylated with 50 mM iodoacetamide (IAA), and incubated for 20 min at 27° C. and 750 rpm on a thermo mixer.

The 96-well hydrophobic PVDF membrane plate (Millipore) was primed with 150 μL of 70% ethanol and equilibrated with 300 μL 8 M urea supernatant and vacuumed through a 96 well plate adaptable vacuum manifold (Millipore). All subsequent liquid transfers were carried out using this 96-well microplate vacuum manifold (Millipore). Each sample was vacuumed three times through the PVDF membrane 96 well plate. After adsorption of the proteins onto the membrane, proteins were washed 2× with 50 mM ammonium bicarbonate. Protein digestion was performed using 1 μg of sequencing grade trypsin (Promega).

To this end, 100 μL digestion buffer (5% acetonitrile ACN, 50 mM ABC and trypsin) were added to each well. After incubation for 2 hours at 37° C. in a humidified incubator, the cleaved proteins, now peptides, were eluted through the vacuum onto a collection 96 well plate. Resulting peptides were eluted using vacuum twice with 150 μL of aqueous 40% ACN containing 0.1% formic acid. Subsequently, the elution solutions were pooled and dried in a vacuum concentrator. Lyophilized samples were resuspended in 50 μL of MS loading buffer (5% FA 5% ACN). Internal retention time standard peptides HRM (Biognosys) were added to the resuspended sample in a 1:10 ratio before introduction to the Thermo Q Exactive mass spectrometer.

### Mass Spectrometric Analysis

A total of 42 urine samples, case (21) and control (21) first point urine biomarker samples were analyzed using a MS Data Dependent Acquisition (DDA) and Data Independent Acquisition (DIA) model on a Q-Exactive mass spectrometer (Thermo Fisher Scientific). A total of 1498 protein groups were identified. A protein group referring to a protein identified by a set of peptides that are unique to that protein.

TABLE 1
Demographics of all patients used in this study

| | All | Control | Neonates | | Regression |
|---|---|---|---|---|---|
| | Neonates | Neonates | with BPD | p-value | p-value |
| Neonates | 42 | 21 | 21 | | |
| (number) | | | | | |
| Gestation | 25.93 | 26.01 | 25.75 | 0.23 | 0.35 |
| age | (±1.18) | (±1.25) | (±1.08) | | |
| (weeks) | | | | | |
| Birth | 809.17 | 853.29 | 764.04 | 0.07 | 0.10 |
| weight | (±175.44) | (±177.39) | (±161.31) | | |
| (g) | | | | | |
| Sex | 18/42 | 11/21 | 7/21 | n/a | 0.22 |
| (# F/# | (43%) | (52%) | (33%) | | |
| total) | | | | | |

### Data and Statistical Analysis

Data Analysis. All DDA and DIA data was analyzed in MaxQuant v 1.6.0.1 (for DDA data) then in Spectronaut Pulsar v13.0 (Biognosys).

Statistical Analysis. Because urinary protein concentration varies with gestational age, we normalized our data set by processing all samples with the same amount of starting volume as mentioned earlier (150 μL of urine). We used Spectronaut to perform a global median normalization using a label free approach to account in our entire dataset for loading differences. We applied the parametric student t-test after log transformation of the intensity values to identify urinary proteins of differential abundance in early samples from ELGANs that later developed BPD (n=21) vs. control ELGANs without BPD (n=21) using Dante R and Perseus v 1.6.10.43. Clinical data was loaded in R-Studio where linear regression model was used to determine if there is a significant difference between the cases and controls for birth weight, gestational age, and sex. The p-value was extracted to determine significance (Table 1). Next, biomarkers and biomarker panel were assessed with the pROC-package where the area under the receiver operating characteristic (AUROC) was calculated and visualized.

### Results

### Characteristics of the Study Population

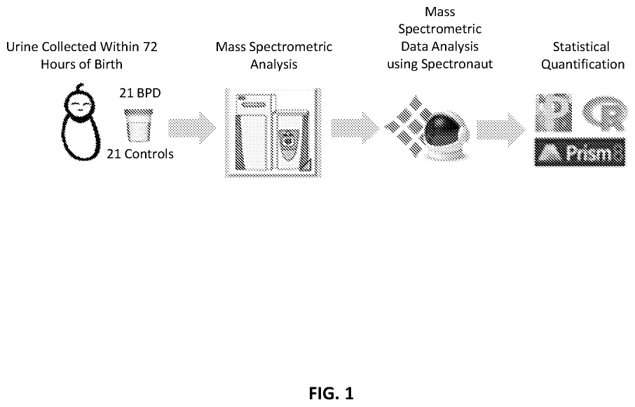

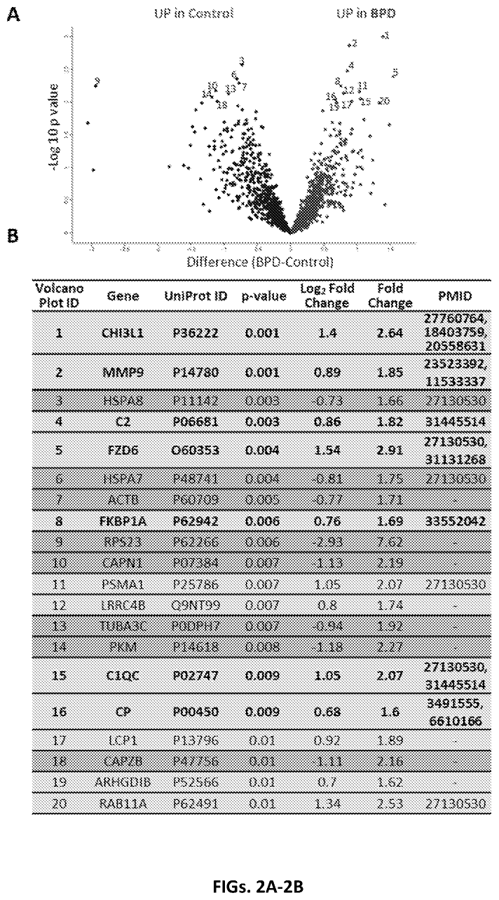

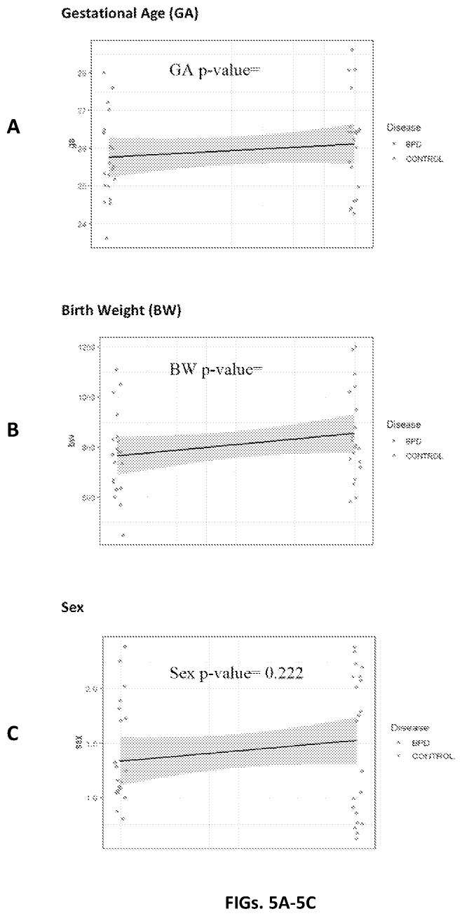

We mapped the proteomes of 42 urine samples collected within 72 hours of birth from 42 ELGANs. The twenty-one ELGANs who later developed BPD (‘cases’) were compared with a similarly sized group who were randomly selected from the study cohort of infants who did not develop BPD (‘controls’). Gestational age, birth weight, and sex did not differ significantly between controls and cases shown in Table 1 and FIGS. 5A-5C. All 42 urine samples were processed using the in-house optimized ‘MStern blotting’ methodology in a single batch in a high throughput manner shown in FIG. 1. We identified and quantified 1498 proteins. Applying a p-value cutoff of 0.05, we identified 94 proteins that showed significant differences in abundance between cases and controls: 35 proteins were up-and 59 proteins were downregulated in BPD patients (shown in FIGS. 2A-2B).

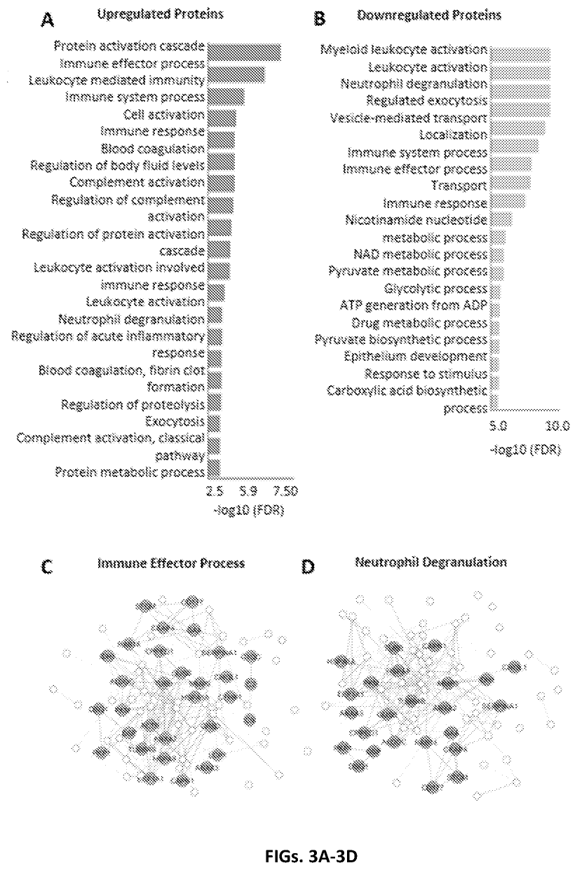

Next, we performed a bioinformatic analysis of these 94 significantly changing proteins using the STRING-based Protein-Protein Interaction Networks Functional Enrichment Analysis (https://string-db.org/). The 35 significantly upregulated proteins were associated with immune effector processes, leukocyte mediated immunity, and complement activation (shown in FIGS. 3A, 3C.) Pathway analysis of the 57 significantly downregulated proteins revealed biological processes related to neutrophil degranulation, leukocyte activation, and various metabolic pathways (shown in FIGS. 3B, 3D). Many of these pathways are associated with inflammation and are well described in the context the pathophysiology of BPD.

Given the exploratory proof-of-concept nature of this study and the small sample size, we concluded that multiple testing correction and/or cross-validations approaches are difficult to implement. Nevertheless, to assess the relevance of the proteins showing statistically significant differences, we performed an extensive literature search with each of the 94 aforementioned proteins. For 20 out of these 94 proteins, we identified references in the context of BPD and/or other pediatric lung pathologies-albeit not in the context of urinary proteins as shown in Table 4. Interestingly, 10 (50%) of these 20 proteins with established link to BPD or other pediatric lung pathologies were found amongst the 20 most significantly changing proteins, while the remaining 10 proteins distributed across the remaining 74 proteins on the list. This literature analysis supports the notion that at least the 20 most significantly changing proteins are highly enriched in BPD-associated proteins.

### Bpd-Associated Changes in Proteins Previously Described Before in Blood or Tracheal Aspirates But not in Urine

Based on this cross-referencing of established literature and our urine proteomics findings, we focused on the 20 proteins with the most significant BPD-associated abundance changes (shown in FIG. 2B). Six of these proteins (CHI3L1, MMP-9, FZD6, CP, CIQC and FKBP1A) have been implicated in either BPD or other lung disease pathophysiology and the remaining 14 proteins are members of specific protein families that have been associated with BPD, such as heatshock and Ras/Rab family proteins (see, e.g., Veerappan A, Thompson M, Savage A R, Silverman M L, Chan W S, Sung B, et al. Mast cells and exosomes in hyperoxia-induced neonatal lung disease. Am J Physiol Lung Cell Mol Physiol. 2016; 310 (11): L1218-32).

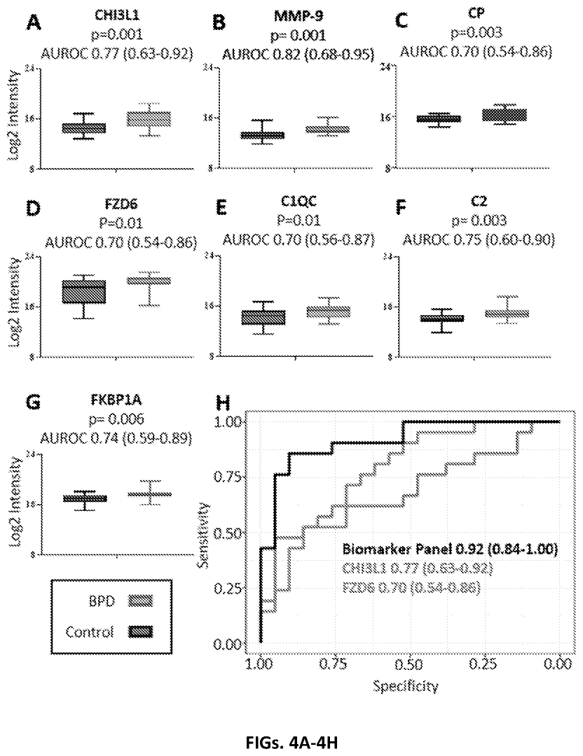

The two proteins found to be most significantly dysregulated were chitinase-3-like protein-1 (CHI3L1, also known as YKL-40) and matrix metalloproteinase-9 (MMP-9). CHI3L1 was measured at a significant 2.6-fold increase in the urine of cases compared to controls (p=0.001) and area under receiver operating characteristic curve (AUROC)=0.77 (95% confidence interval (CI): 0.63-0.92) (shown in FIG. 4A). MMP-9 showed a significant almost 3-fold increase in the urine of the cases compared to controls (p=0.001, AUROC=0.82 (95% CI: 0.68-0.95)) (shown in FIG. 4B).

Additional proteins with known roles in BPD pathophysiology included ceruloplasmin (CP), frizzled-6 (FZD6), complement pathway components, and peptidyl-prolyl cis-trans isomerase (FKBP1A). CP, a well characterized protein with an important role in iron metabolism (see, e.g., Collard K J. Iron homeostasis in the neonate. Pediatrics. 2009; 123 (4): 1208-16), was 1.6-fold upregulated in the cases vs. controls (p=0.003, AUROC=0.70 (95% CI: 0.54-0.86) (as shown in FIG. 4C). This was followed by a 2.9-fold upregulation of FZD6, a protein in the canonical WNT pathway and a main player in embryological pulmonary system development (p=0.01, AUROC 0.70 (95% CI 0.54-0.86) (as shown in FIG. 4D). Thirdly, we also identified Complement Clq subcomponent subunit C (C1QC) and Complement C2 (C2), i.e., initial components of the classical and lectin complement pathways. While these proteins have not previously been reported to be dysregulated in BPD, the complement pathways have been (see, e.g., Suski M, Bokiniec R, Szwarc-Duma M, Madej J, Bujak-Gizycka B, Borszewska-Kornacka M K, et al. Plasma proteome changes in cord blood samples from preterm infants. J Perinatol. 2018; 38 (9): 1182-9). C1QC (p=0.010, AUROC 0.71 (95% CI: 0.56-0.87)) and C2 (p=0.003, AUROC 0.75 (95% CI: 0.60-0.90)) were both significantly upregulated in the cases vs. controls (shown in FIGS. 4E, 4F). Finally, FKBPIA, which is known to play a role in hypoxia-related pulmonary hypertension, immune modulation, and recently in post-prematurity respiratory disease (see, e.g., Bhattacharya S, Mereness J A, Baran A M, Misra R S, Peterson D R, Ryan R M, et al. Lymphocyte-Specific Biomarkers Associated With Preterm Birth and Bronchopulmonary Dysplasia. Frontiers in Immunology. 2021; 11), showed a significant 2-fold upregulation in cases (p=0.006, AUROC=0.73 (95% CI: 0.59-0.89)) (shown in FIG. 4G).

We investigated the possibility of characterizing a urinary protein panel to identify ELGANs during the first three days of life at risk for developing BPD. This analysis was performed by using 1) the list of proteins previously validated in BPD (Table 5) and 2) the list of proteins with the most significant BPD-associated abundance changes (shown in FIG. 2B). We used logistic regression to identify a 2-protein panel comprising of CHI3L1 and FZD6 (AUROC=0.92 (95% CI: 0.84-1.00) (shown in FIG. 4H), which have both been validated in BPD literature, and a 3-protein panel comprising MMP-9, TUBA3C, and CAPZB (AUROC=0.94 (95% CI: 0.86-1.00) and p<0.0001), shown in FIGS. 5A-5C, which may have increased predictability for the development of BPD.

### Dysregulated Urinary Proteins Identify Potential FDA-Approved Drug Targets in BPD

Finally, our results identified a total of 16 proteins with significant abundance differences as shown in Table 2 that are known targets of Food and Drug Administration (FDA)-approved drug. Of those 16 proteins, six have been previously associated with development of BPD. These six proteins include in addition to the above described MMP-9 (target of marimastat and captopril) and FKBP1A (target of tacrolimus and pimecrolimus) the following four proteins: i) Lysosomal alpha-glucosidase (GAA), which is inhibited by miglitol (see, e.g., Kato A, Nakagome I, Hata M, Nash R J, Fleet G W J, Natori Y, et al. Strategy for Designing Selective Lysosomal Acid alpha-Glucosidase Inhibitors: Binding Orientation and Influence on Selectivity. Molecules. 2020; 25 (12)); ii) Collagen alpha-1 (I) chain (COL1A1), which was found to be increased in lung tissue of BPD patients (see, e.g., Togari H, Hashimoto Y, Wada Y, Hayakawa T. Increased type III/I collagen and alpha 1 (I)/alpha 2 (I) chain in a bronchopulmonary dysplastic lung. Acta Paediatr Jpn. 1993;35 (2): 101-7) is inhibited by the drug halofuginone used to treat scleroderma, cancer, and restenosis (see, e.g., Elkin M, Miao H Q, Nagler A, Aingorn E, Reich R, Hemo I, et al. Halofuginone: a potent inhibitor of critical steps in angiogenesis progression. FASEB J. 2000;14 (15): 2477-85); iii) Carbonic anhydrase 2 (CA2) has been described as a target of a) the diuretic bendroflumethiazide, which has been used in neonates with developing chronic lung disease, and b) celecoxib, a drug used to reduce pain and inflammation; iv) Annexin 3 (ANXA3), implicated in BPD disease progression, is indirectly inhibited by the drug diflucorolone used to reduce inflammation in a variety of inflammatory autoimmune skin disorders such as eczema and psoriasis (see, e.g., Uva L, Miguel D, Pinheiro C, Antunes J, Cruz D, Ferreira J, et al. Mechanisms of action of topical corticosteroids in psoriasis. Int J Endocrinol. 2012; 2012:561018).

TABLE 2
List of 16 significant proteins identified as FDA approved
drug targets. Each protein is referenced with ChEMBL

| | | | Fold | | |
|---|---|---|---|---|---|
| Gene | UniProt ID | p-value | Change | ChEMBL ID | DrugBank Database |
| MMP9 | P14780 | 0.001 | 1.85 | CHEMBL321 | Marimastat, Captopril |
| FKBP1A | P62942 | 0.006 | 1.69 | CHEMBL1902 | Tacrolimus, |
| | | | | | Pimecrolimus |
| CA2 | P00918 | 0.021 | 1.68 | CHEMBL205 | Bendroflumethiazide, |
| | | | | | Celecoxib |
| GAA | P10253 | 0.022 | 0.62 | CHEMBL2608 | Miglitol |
| SLC12A1 | Q13621 | 0.021 | 0.63 | CHEMBL1874 | Bumetanide |
| TUBB | P07437 | 0.025 | 0.74 | CHEMBL5444 | Artenimol |
| LDHA | P00338 | 0.025 | 0.41 | CHEMBL4835 | Stiripentol |
| GAPDH | P04406 | 0.026 | 0.51 | CHEMBL2284 | Artenimol |
| LCK | P06239 | 0.040 | 0.45 | CHEMBL258 | Dasatinib |
| COL1A1 | P02452 | 0.040 | 1.75 | CHEMBL2364188 | Halofuginone |
| TFPI | P10646 | 0.043 | 1.68 | CHEMBL3713062 | Dalteparin |
| LDHB | P07195 | 0.045 | 0.65 | CHEMBL4940 | Artenimol |
| EGF | P01133 | 0.045 | 0.72 | CHEMBL5734 | Cholecystokinin |
| TUBB4B | P68371 | 0.047 | 0.69 | CHEMBL1848 | Oxibendazole |
| EPHA2 | P29317 | 0.049 | 0.63 | CHEMBL2068 | Dasatinib |
| ANXA3 | P12429 | 0.050 | 0.61 | N/A | Diflucorolone |

### Panels

The present invention provides two urinary protein-based biomarker panels that are found to be useful in identifying premature neonates at risk for developing Bronchopulmonary Dysplasia (BPD), a debilitating lung disease. These biomarker panels were derived from utilizing our in house developed high throughput urine proteomics methodology that requires as little as 50 microliters of urine. We utilized our methodology to validate proteins previously identified in invasive samples such as blood and/or tracheal aspirates on urine collected within 72 hours of birth from Extremely Low Gestational Age Neonates (ELGANs) (gestational age (26+1.2) weeks) who were admitted to a single Neonatal Intensive Care Unit (NICU); half of whom eventually developed BPD (n=21), while the other half served as controls (n=21). Our high throughput urine proteomics approach clearly identified several BPD-associated changes in the urine proteome recapitulating expected blood proteome changes and several urinary proteins predicted BPD risk. Interestingly, sixteen of our identified urinary proteins are known targets of drugs approved by the Food and Drug Administration. In addition to validating numerous proteins, previously found in invasively collected blood, tracheal aspirate, and broncho-alveolar lavage, that have been implicated in BPD pathophysiology, urine proteomics also suggested novel potential therapeutic targets. Ease of access to urine could allow for sequential proteomic evaluations for longitudinal monitoring of disease progression and impact of therapeutic intervention.

### Panel 1:

We used logistic regression to identify a 2-protein panel comprising of CHI3L1 and FZD6 (AUROC=0.92 (95% CI: 0.84-1.00)). This panel consists of the proteins chitinase-3-like protein-1 (CHI3L1, also known as YKL-40) and Frizzled-6 (FZD6). CHI3L1/YKL-40 is known as a potential screening biomarker for extremely preterm infants with BPD who are at risk for developing pulmonary hypertension. CHI3L1/YKL-40 is also detected in cord blood to predict the onset of asthma (see, e.g., Al-Ghanem G, Shah P, Thomas S, Banfield L, El Helou S, Fusch C, et al. Bronchopulmonary dysplasia and pulmonary hypertension: a meta-analysis. J Perinatol. 2017;37 (4): 414-9). CHI3L1/YKL-40 protein plays a highly lung specific role in tissue remodeling, inflammation, hyperoxia induced injury, epithelial apoptosis (see, e.g., Siffel C, Kistler K D, Lewis J F M, Sarda S P. Global incidence of bronchopulmonary dysplasia among extremely preterm infants: a systematic literature review. J Matern Fetal Neonatal Med. 2019:1-11), and as a protective factor in respiratory infections. FZD6, a protein in the canonical WNT pathway and a main player in embryological pulmonary system development. In short, while both proteins have been described before in the context of BPD or other lung pathologies of (pre-term) neonates, they have never been described as being dysregulated in urine, which would be the non-obvious extension of the prior work.

### Panel 2:

Panel two consists of three proteins matrix metalloproteinase-9 (MMP-9), Tubulin Alpha 3C (TUBA3C), and F-actin-capping protein subunit beta (CAPZB). This 3-protein panel, identified by logistic regression reached an AUROC of 0.94 (95% CI: 0.86-1.00). MMPs are proteinases, which also play an important role in lung remodeling and lung development, and are strongly associated with BPD (see, e.g., Kentsis A, Ahmed S, Kurek K, Brennan E, Bradwin G, Steen H, et al. Detection and diagnostic value of urine leucine-rich alpha-2-glycoprotein in children with suspected acute appendicitis. Ann Emerg Med. 2012; 60 (1): 78-83 e1). An increase in MMP-9 has been identified in tracheal aspirate fluid of preterm infants and MMP-9 levels are correlated with increased BPD incidence and severity of BPD which is consistent with our observation that ELGAN cases manifested increased urinary MMP-9 levels. It is worth noting, that MMP-9 is a well-described target of the FDA-approved drug marimastat and captopril, which are used as a broad range MMP inhibitor for the treatment of various cancers (see, e.g., Berger S T, Ahmed S, Muntel J, Cuevas Polo N, Bachur R, Kentsis A, et al. MStern Blotting-High Throughput Polyvinylidene Fluoride (PVDF) Membrane-Based Proteomic Sample Preparation for 96-Well Plates. Mol Cell Proteomics. 2015;14 (10): 2814-23) and for the treatment of hypertension (see, e.g., Kentsis A, Shulman A, Ahmed S, Brennan E, Monuteaux M C, Lee Y H, et al. Urine proteomics for discovery of improved diagnostic markers of Kawasaki disease. EMBO Mol Med. 2013; 5 (2): 210-20), respectively. Tubulin Alpha 3C (TUBA3C), and F-actin-capping protein subunit beta (CAPZB) are novel BPD-associated proteins identified in our study. Tables 3 and 4 list protein markers and public information on drug targeting the markers.

TABLE 3
Protein markers and public information on drug targeting the markers

| | UniProt | | ChEMBL | DrugBank | Generic? |
|---|---|---|---|---|---|
| Gene | ID | Protein Name | ID | Database | (Y/N) |
| MMP9 | P14780 | Matrix | CHEMBL321 | Marimastat, | Y |
| | | metalloproteinase-9 | | Captopril | |
| FKBP1A | P62942 | Peptidyl-prolyl cis-trans | CHEMBL1902 | Tacrolimus, | Y |
| | | isomerase FKBP1A | | Pimecrolimus | |
| CA2 | P00918 | Carbonic anhydrase 2 | CHEMBL205 | Bendroflumethiazide, | Y |
| | | | | Celecoxib | |
| GAA | P10253 | Lysosomal alpha- | CHEMBL2608 | Miglitol | Y |
| | | glucosidase | | | |
| SLC12A1 | Q13621 | Solute carrier family 12 | CHEMBL1874 | Bumetanide | Y |
| | | member 1 | | | |
| TUBB | P07437 | Tubulin beta chain | CHEMBL5444 | Artenimol | Y |
| LDHA | P00338 | L-lactate dehydrogenase | CHEMBL4835 | Stiripentol | Aug. 20, 2023 |
| | | A chain | | | |
| GAPDH | P04406 | Glyceraldehyde-3- | CHEMBL2284 | Artenimol | y |
| | | phosphate dehydrogenase | | | |
| LCK | P06239 | Tyrosine-protein kinase | CHEMBL258 | Dasatinib | y |
| | | Lck | | | |
| COL1A1 | P02452 | Collagen alpha-1(I) chain | CHEMBL2364188 | Halofuginone | veterinary |
| | | | | | drug |
| TFPI | P10646 | Tissue factor pathway | CHEMBL3713062 | Dalteparin | May 16, 2022 |
| | | inhibitor | | | |
| LDHB | P07195 | L-lactate dehydrogenase | CHEMBL4940 | Artenimol | Y |
| | | B chain | | | |
| EGF | P01133 | Pro-epidermal growth | CHEMBL5734 | Cholecystokinin | Aug. 16, 2022 |
| | | factor | | (Sincalide) | |
| TUBB4B | P68371 | Tubulin beta-4B chain | CHEMBL1848 | Oxibendazole | veterinary |
| | | | | | drug |
| EPHA2 | P29317 | Ephrin type-A receptor 2 | CHEMBL2068 | Dasatinib | Y |
| ANXA3 | P12429 | Annexin A3 | N/A | Diflucorolone | not listed |
| | | | | | in FDA OB; |
| | | | | | on sale in |
| | | | | | Japan |

TABLE 4
Selected significant proteins related to BPD

| | | | | Log2 | |
|---|---|---|---|---|---|
| | | | −LOG10 | Fold | |
| | Protein Name | Accessions | (P-value) | Change | PMID(s) |
| 1 | Chitinase-3-like | P36222 | 3.00 | 1.40 | 27760764; |
| | protein 1 | | | | 33498968; |
| | | | | | 32312371; |
| | | | | | 32772428; |
| | | | | | 20558631; |
| | | | | | 26353077 |
| 2 | Matrix | P14780 | 2.87 | 0.89 | 26779482; |
| | metalloproteinase-9 | | | | 11533337; |
| | | | | | 12376362; |
| | | | | | 14973177; |
| | | | | | 15521085; |
| | | | | | 18336743; |
| | | | | | 19026401; |
| | | | | | 19097983; |
| | | | | | 19205055; |
| | | | | | 21216975; |
| | | | | | 18676410; |
| | | | | | 26353077 |
| 3 | Complement C2 | P06681 | 2.48 | 0.86 | 31445514; |
| | | | | | 26353077 |
| 4 | Frizzled-6 | O60353 | 2.38 | 1.54 | 31131268 |
| 5 | Peptidyl-prolyl cis- | P62942 | 2.25 | 0.76 | 33552042; |
| | trans isomerase | | | | 24981452 |
| | FKBP1A | | | | |
| 6 | Leucine-rich repeat- | Q9NT99 | 2.13 | 0.80 | 31445514 |
| | containing protein | | | | |
| | 4B | | | | |
| 7 | Complement C1q | P02747 | 2.05 | 1.05 | 31445514; |
| | subcomponent | | | | 26353077 |
| | subunit C | | | | |
| 8 | Ceruloplasmin | P00450 | 2.03 | 0.68 | 3491555; |
| | | | | | 6610166; |
| | | | | | 10970997; |
| | | | | | 8293704; |
| | | | | | 9713035 |
| 9 | Carboxypeptidase M | P14384 | 1.74 | −0.53 | 31445514 |
| 10 | Coagulation factor | P05160 | 1.73 | 1.10 | 23709621 |
| | XIII B chain | | | | |
| 11 | Carbonic | P00918 | 1.68 | 0.75 | 23709621 |
| | anhydrase 2 | | | | |
| 12 | Lysosomal alpha- | P10253 | 1.66 | −0.70 | 9877121 |
| | glucosidase | | | | |
| 13 | Complement factor | Q03591 | 1.58 | 0.52 | 31445514; |
| | H-related protein 1 | | | | 26353077 |
| 14 | Complement | P02748 | 1.52 | 0.92 | 31445514 |
| | component C9 | | | | |
| 15 | Immunoglobulin | P01706 | 1.49 | 0.61 | 31445514 |
| | lambda variable | | | | |
| | 2-11 | | | | |
| 16 | Collagen alpha-1(I) | P02452 | 1.40 | 0.81 | 8503265 |
| | chain | | | | |
| 17 | Fibulin-1 | P23142 | 1.36 | 0.47 | 26779482 |
| 18 | Immunoglobulin | P0DP02 | 1.36 | 0.82 | 31445514 |
| | heavy variable | | | | |
| | 3-30-3 | | | | |
| 19 | Alpha-1-antitrypsin | P01009 | 1.35 | 0.76 | 21705962; |
| | | | | | 10460951; |
| | | | | | 31818194 |
| 20 | Annexin A3 | P12429 | 1.30 | −0.71 | 27065351 |

### DISCUSSION

The primary objective of this study was to show that it is feasible to use urine samples from preterm infants collected during the first three days of life to confirm the dysregulation of proteins already known to be associated with BPD and to provide novel insights into BPD pathophysiology. We analyzed urine samples that were collected within 72 hours of birth, i.e., within the critical first 3 days of early phase BPD, using a non-invasive and easy method compatible with the NICU standard of care: cotton balls placed in the diaper. Our findings were fully consistent with BPD literature on proteins identified from invasively collected body fluids such as blood, tracheal aspirate, and broncho-alveolar lavage.

We would like to highlight the fact that our study is in many ways an orthogonal validation study by itself, as we focus on the proteins that have been described before as being dysregulated in the context of BPD and/or neonatal lung disorders, albeit not in the context of urinary proteome components and not necessarily using LC/MS as detection and quantification method. Previous urine proteomic studies have successfully identified disease-associated changes in the urinary proteome for various childhood diseases. However, these studies required 50-times larger starting volumes, and used laborious and time-consuming sample preparation methods. Also, despite the small amount of urine used (50 μL), a comprehensive urinary proteome with >1400 proteins were mapped. Given that we identified a similar number of proteins in urine from ELGANs as in urine from toddlers and pre-teens, it can be concluded that our urine proteomics pipeline is fully compatible with miniscule urine samples that can be routinely and non-invasively collected in NICUs.

Additionally, we see major dysregulation of proteins associated with immune effector processes including complement pathway proteins, which is consistent with a previous animal study that identified increased expression of complement components in a newborn rat lung injury model (see, e.g., Denervaud V, Gremlich S, Trummer-Menzi E, Schittny J C, Roth-Kleiner M. Gene expression profile in newborn rat lungs after two days of recovery of mechanical ventilation. Pediatr Res. 2015;78 (6): 641-9). Of interest, blood coagulation and fibrin clot formation pathways were associated with proteins with statistically significant changes in abundance in our urine proteomics study. In agreement, dysregulated coagulation and fibrinolytic activity have been described when analyzing lung lavage fluid from BPD patients (see, e.g., Viscardi R M, Broderick K, Sun C C, Yale-Loehr A J, Hessamfar A, Taciak V, et al. Disordered pathways of fibrin turnover in lung lavage of premature infants with respiratory distress syndrome. Am Rev Respir Dis. 1992; 146 (2): 492-9). Neutrophil degranulation, which was also identified in our analysis, is a common characteristic of many inflammatory responses including septic shock, asthma, acute lung injury, and BPD when analyzing tracheal aspirates (see, e.g., Genschmer K R, Russell D W, Lal C, Szul T, Bratcher P E, Noerager B D, et al. Activated PMN Exosomes: Pathogenic Entities Causing Matrix Destruction and Disease in the Lung. Cell. 2019; 176 (1-2): 113-26 e15). It is assumed that a dysregulation of proinflammatory cytokines produced by alveolar macrophages and changes in neutrophil counts contribute to persistent lung inflammation (see, e.g., Shahzad T, Radajewski S, Chao C M, Bellusci S, Ehrhardt H. Pathogenesis of bronchopulmonary dysplasia: when inflammation meets organ development. Mol Cell Pediatr. 2016; 3 (1): 23). In summary, the bioinformatic analysis of the dysregulated urine proteins accurately reflects many known pathways related to BPD pathophysiology.

We identified multiple proteins that have been described before in the context of BPD such as CHI3L1, MMP-9, and CP. However, those reports used blood or tracheal aspirate/BAL fluid but never urine. One previous study investigated CHI3L1/YKL-40 as a potential screening biomarker for extremely preterm infants with BPD who are at risk for developing pulmonary hypertension (see, e.g., Konig K, Guy K J, Nold-Petry C A, Barfield C P, Walsh G, Drew S M, et al. BNP, troponin I, and YKL-40 as screening markers in extremely preterm infants at risk for pulmonary hypertension associated with bronchopulmonary dysplasia. Am J Physiol Lung Cell Mol Physiol. 2016; 311 (6): L1076-L81). Another study used cord blood to detect CHI3L1/YKL-40 to predict the onset of asthma (see, e.g., Ober C, Tan Z, Sun Y, Possick J D, Pan L, Nicolae R, et al. Effect of variation in CHI3L1 on serum YKL-40 level, risk of asthma, and lung function. N Engl J Med. 2008;358 (16): 1682-91). CHI3L1/YKL-40 protein plays a highly lung specific role in tissue remodeling, inflammation, hyperoxia induced injury, epithelial apoptosis (see, e.g., Sohn M H, Kang M J, Matsuura H, Bhandari V, Chen N Y, Lee C G, et al. The chitinase-like proteins breast regression protein-39 and YKL-40 regulate hyperoxia-induced acute lung injury. Am J Respir Crit Care Med. 2010; 182 (7): 918-28.), and as a protective factor in respiratory infections. Overall, our results are consistent with published literature and, to our knowledge, ours is the only study thus far detecting increased levels of CHI3L1/YKL-40 noninvasively in urine of ELGANs prior to developing BPD.

MMPs are proteinases, which also play an important role in lung remodeling and lung development, and are strongly associated with BPD (see, e.g., Shahzad T, Radajewski S, Chao C M, Bellusci S, Ehrhardt H. Pathogenesis of bronchopulmonary dysplasia: when inflammation meets organ development. Mol Cell Pediatr. 2016; 3 (1): 23). An increase in MMP-9 has been identified in tracheal aspirate fluid of preterm infants and MMP-9 levels are correlated with increased BPD incidence and severity of BPD which is consistent with our observation that ELGAN cases manifested increased urinary MMP-9 levels. It is worth noting, that MMP-9 is a well-described target of the FDA-approved drug marimastat and captopril, which are used as a broad range MMP inhibitor for the treatment of various cancers (see, e.g., Underwood C K, Min D, Lyons J G, Hambley T W. The interaction of metal ions and Marimastat with matrix metalloproteinase 9. J Inorg Biochem. 2003; 95 (2-3): 165-70) and for the treatment of hypertension (see, e.g., Okada M, Kikuzuki R, Harada T, Hori Y, Yamawaki H, Hara Y. Captopril attenuates matrix metalloproteinase-2 and -9 in monocrotaline-induced right ventricular hypertrophy in rats. J Pharmacol Sci. 2008;108 (4): 487-94), respectively.

Ceruloplasmin is a copper-binding glycoprotein that has ferroxidase activity oxidizing Fe2+ to Fe3+ without releasing radical oxygen species, i.e., change in this enzymatic activity could be associated with the dysregulated iron metabolism, which is a known complication in preterm infants at risk for BPD. Of note, elevated levels of serum ceruloplasmin have been associated with respiratory distress syndrome (RDS), a prerequisite for BPD (see, e.g., Rosenfeld W, Concepcion L, Evans H, Jhaveri R, Sahdev S, Zabaleta I. Serial trypsin inhibitory capacity and ceruloplasmin levels in prematures at risk for bronchopulmonary dysplasia. Am Rev Respir Dis. 1986;134 (6): 1229-32).

Inflammation plays a crucial role in BPD pathophysiology. A downregulation of complement proteins Cls and Clr, and upregulation of C3, were detected in premature infant cord blood in the context of BPD in a previous study (see, e.g., Suski M, Bokiniec R, Szwarc-Duma M, Madej J, Bujak-Gizycka B, Borszewska-Kornacka M K, et al. Plasma proteome changes in cord blood samples from preterm infants. J Perinatol. 2018; 38 (9): 1182-9). Additionally, C1 subunits were also identified as a biomarker for BPD in plasma. In our cohort, urinary C2 and C1QC were upregulated in neonates who later developed BPD. These data support the important role of the complement system in BPD, and with our study being the first to identify these changes specifically in urine of pre-BPD infants.

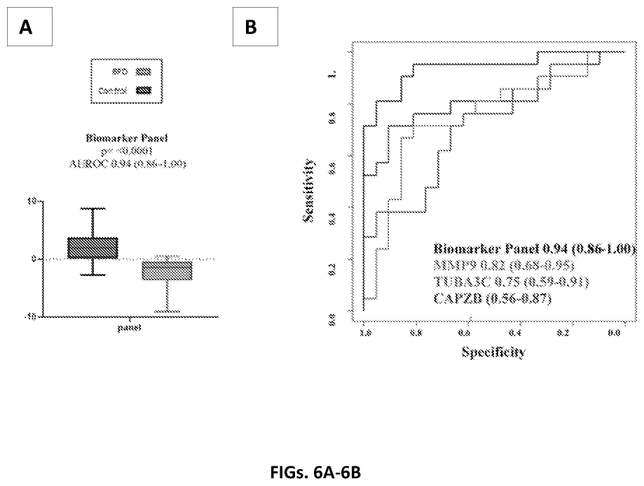

Mining the differentially expressed urinary proteins allowed us to propose a promising candidate 2-protein biomarker panel (CHI3L1 and FZD6; FIG. 4H) based on previously validated BPD-associated proteins. We also identified a 3-protein biomarker panel (MMP-9, TUBA3C, and CAPZB), based on previously validated BPD-associated proteins (MMP-9) and novel BPD-associated proteins (TUBA3C and CAPZB), with increased performance based on AUROC as shown in FIGS. 6A-6B that may identify the ELGAN neonates at risk for BPD.

FKBPIA, known to play a role in hypoxia-related pulmonary hypertension (see, e.g., Kwapiszewska G, Wilhelm J, Wolff S, Laumanns I, Koenig I R, Ziegler A, et al. Expression profiling of laser-microdissected intrapulmonary arteries in hypoxia-induced pulmonary hypertension. Respir Res. 2005; 6:109), was upregulated in the BPD cohort, consistent with the increased risk of pulmonary hypertension in neonate with BPD. We identified 16 targets for FDA-approved drugs including two proteins (MMP-9 and FKBP1A) that were amongst our top 20 significantly changing urinary proteins, thereby highlighting urine proteomics as a strategy to identify potential novel therapeutic approaches to treat BPD. In this context, it is interesting to note that at least four of the drugs (halofuginone, difluocortolone, tacrolimus, pimecrolimus) linked to three of our significant proteins are widely used for inflammatory skin conditions associated with remodeling, abnormal growth and/or fibrosis. This finding suggests that non-topical preparations of these drugs are potentially a promising starting point for the development of novel therapeutic approaches for BPD.

Put together, our results reflect proteins involved in lung pathophysiology although unclear if inflammation at the lung site and/or abnormal pulmonary development caused by dysregulated proteins result in the clinical presentation associated with BPD. Similarly, the cross-talk in the immune-modulatory plasma and urine proteomes is not yet fully understood. Therefore, there are a number of limitations in our study: First, we were not able to perform sequential proteomic analysis in our cohort, with matching plasma and urine samples collected within the same hour, as this was a pilot proof of concept and an independent validation cohort study is still required. Second, in some cases, prenatal factors (e.g., chorioamnionitis (Baud O, Laughon M, Lehert P. Survival without Bronchopulmonary Dysplasia of Extremely Preterm Infants: A Predictive Model at Birth. Neonatology. 2021; 118 (4): 385-93) as well as postnatal factors would have played a role in the development of BPD. However, the majority of patients born in this cohort (<29 weeks' GA) were exposed to prenatal steroids and/or received postnatal antibiotics, hence future studies will need to be designed to investigate the effect of these variables. Third, the sample size does not allow for sensitivity analysis of characteristics such as ethnicity and race. Our overall goal is to be able to identify proteins significant regardless of these variables. However, future studies with larger cohorts could explore how co-morbidities, prenatal and post-natal management, and race and ethnicity affected the levels of these proteins.

In conclusion, our study does show that disease-associated changes in the urine proteome closely resemble the molecular changes that have been described for blood, tracheal aspirate, and broncho-alveolar lavage. This is advantageous as it highlights a non-invasive monitoring platform for BPD-associated proteins that can be used for investigating BPD pathogenesis, morbidity, and mortality. In the future, using urine proteomics to discover new proteins and biological pathways underlying BPD development could be used 1) to tailor novel precision medicine approaches for the treatment of this serious disease that has life-long consequences and 2) as a predictor to select high BPD risk cohorts for prevention and intervention clinical trials.

### Supplemental Materials and Methods

### Study Population and Urine Sample Collection

This study was designed as a proof of concept. All urine samples were obtained from ELGANs admitted to the Neonatal Intensive Care Unit (NICU) at Brigham and Women's Hospital (BWH), Boston, Massachusetts, between 1997 and 2015 through a Partners Human Research Committee-approved discarded specimen and medical records protocol. The BWH Institutional Review Board granted an exemption from requiring written informed consent. All infants in the BWH cohort were born before 29 weeks of gestation. Most patients in this gestational age (GA) cohort (<29 weeks' GA) were exposed to prenatal steroids and/or received postnatal antibiotics and standard nutritional management including parenteral nutrition as clinically indicated. Comorbidities are described in FIG. 7. BPD cases were defined as infants requiring supplemental oxygen at 36 weeks' postmenstrual age (PMA). BPD cases were compared with controls from the remaining cohort who did not meet this definition of BPD. Race/Ethnicity of the 42 samples are described in FIG. 8. Urine was collected within 72 hours of birth by placing a cotton ball in the baby's diaper and then extracting the urine by compressing the wet cotton ball into the barrel of a plastic syringe and pressing down on the plunger until no fluid remained. The urine was centrifuged to remove any remaining debris, aliquoted into Eppendorf tubes and stored at −80° C.

### Mass Spectrometric Analysis

Data Dependent Acquisition (DDA). A total of 42 urine samples, case (21) and control (21) first point urine biomarker samples were analyzed using a data-dependent TOP10 acquisition method by initial liquid chromatography separation by using a microfluidic chip system (Eksigent, trapping column: 200 μm×0.5 mm ReproSil-Pur C18-AQ 3 μm; analytical column: 75 μm×15 cm ReproSil-Pur C₁₈-AQ 3 μm) followed by DDA analysis on a Q-Exactive mass spectrometer (Thermo Fisher Scientific). Peptides were separated by a linear gradient from 93% buffer A (0.2% FA in HPLC water)/7% buffer B (0.2% FA in ACN) to 75% buffer A/25% buffer B within 75 min at 1000 nL/min. The mass spectrometer was operated in data-dependent TOP10 mode with the following settings: mass range 400-1000 Th; resolution for MS1 scan 70 000 @ 200 Th; lock mass: 445.120025 Th; resolution for MS2 scan 17 500 @ 200 Th; isolation width 1.6 Th; NCE 27; underfill ratio 1%; charge state exclusion: unassigned, 1, >6; dynamic exclusion 30 sec. Resulting DDA data for was used for MS1 level quantitation as well as generation of a spectra library.

Data Independent Acquisition (DIA). A total of 42 urine samples, case (21) and control (21) first point urine biomarker samples were analyzed using a MS DIA acquisition method by initial liquid chromatography separation by using a microfluidic chip system (Eksigent, trapping column: 200 μm×0.5 mm ReproSil-Pur C₁₈-AQ 3 μm; analytical column: 75 μm×15 cm ReproSil-Pur C₁₈-AQ 3 μm) followed by DIA analysis on a Q-Exactive mass spectrometer (Thermo Fisher Scientific). Peptides were separated by a linear gradient from 93% buffer A (0.2% FA in HPLC water)/7% buffer B (0.2% FA in ACN) to 75% buffer A/25% buffer B within 75 min. DIA samples on the Q-Exactive mass spectrometer (Thermo Fisher Scientific) were performed with the same LC setup and gradient as DDA method but instead using a DIA method with following settings: 1 full MS scan with 17,500 resolution at 200 Th; AGC target-3e6; maximum injection time-80 ms; scan range −400 to 1,000 Th; followed by a DIA scan event at 17,500 resolution at 200 Th; AGC target −1e6; maximum IT-auto; loop count-15; MSX count 1; isolation width-20 Th; fixed first mass-200 Th; nCE-27; covering a mass range from 400 to 700 Th. Then, an additional full MS scan with the same parameters was used, followed by an additional DIA scan event with the same parameters covering a mass range from 700 to 1,000 Th. In total a mass range from 400-1,000 Th was covered. Resulting DIA data was used for MS2 level quantitation by searching against DDA generated spectra library. A total of 1498 protein groups were identified. A protein group referring to a protein identified by a set of peptides that are unique to that protein.

### Data and Statistical Analysis

Spectral Library Construction. All DDA.raw files were directly processed with MaxQuant v 1.6.0.1 (see, e.g., Al-Ghanem G, Shah P, Thomas S, Banfield L, El Helou S, Fusch C, et al. Bronchopulmonary dysplasia and pulmonary hypertension: a meta-analysis. J Perinatol. 2017;37 (4): 414-9) using Uniprot Human reference proteome with iRT peptide sequence added (Biognosys). Standard settings were used in MaxQuant with the following modifications: carbamidomethylated cysteine residues (fixed), acetylation of the N-terminal of proteins (variable), oxidation of methionine (variable). Match between runs analysis was enabled and only the filtered 1% FDR identifications were used. A spectral library was generated in Spectronaut Pulsar v 13.0 (Biognosys) using default settings.

DIA Data Analysis. All DIA data was analyzed directly in Spectronaut Pulsar v13.0 (Biognosys). Standard settings were employed, which included dynamic peak detection, automatic precision non-linear iRT calibration, interference correction, and cross run normalization enabled. Spectronaut utilizes the spiked-in HRM peptides for m/z and retention time calibration. All results were filtered by a sparse q-value of 0.01 (equal to FDR of 1% on peptide level). All other settings were set to default. All statistical analysis was performed using Spectronaut Pulsar v 13.0.

Statistical Analysis. Because urinary protein concentration varies with gestational age, we did not use creatinine (Cr) or urine output to normalize, but rather used a sample processing and bioinformatics approach to normalize our entire data set, as we did not have information about urine creatinine levels and/or urine output in this cohort. Instead, we normalized our data set by processing all samples with the same amount of starting volume as mentioned earlier (150 μL of urine). During post processing of our mass spec data, we used a commercial software Spectronaut to perform a global median normalization using a label free approach to account in our entire dataset for loading differences. We applied the parametric student t-test after log transformation of the intensity values to identify urinary proteins of differential abundance in early samples from ELGANs that later developed BPD (n=21) vs. control ELGANs without BPD (n=21) using Dante R and Perseus v 1.6.10.43). Additionally, clinical data was loaded in R-Studio where linear regression model was used to determine if there is a significant difference between the cases and controls for birth weight, gestational age, and sex. The quantified protein matrix was loaded into R using R-studio for biomarker panel development. In R the data was prepared using Tidyverse-packages following by logistic regression analysis using the bestglm-package (see, e.g., Lal C V, Bhandari V, Ambalavanan N. Genomics, microbiomics, proteomics, and metabolomics in bronchopulmonary dysplasia. Semin Perinatol. 2018; 42 (7): 425-31) where Bayesian Information Criterion was used to select the optimal model. Next, biomarkers and biomarker panel were assessed with the pROC-package where the area under the receiver operating characteristic (AUROC) was calculated and visualized. The p-value was extracted to determine significance. Data was visualized using the ggplot2-package (FIGS. 5A-5C).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. (canceled)

2. A method of detecting a pulmonary disease or disorder in a subject, comprising
(a) obtaining a urine sample from the subject;
(b) detecting the abundance of one or more proteins selected from the group consisting of CHI3L1 (UniProt ID P36222), MMP9 (UniProt ID P14780), HSPA8 (UniProt ID P111420, C2 (UniProt ID P06681), FZD6 (UniProt ID 060353), HSPA7 (UniProt ID P48741), ACTB (UniProt ID P60709), FKBPIA (UniProt ID P62942), RPS23 (UniProt ID P62266), CAPNI (UniProt ID P07384), PSMA1 (UniProt ID P25786), LRRC4B (UniProt ID Q9NT99), TUBA3C (UniProt ID PODPH7), PKM (UniProt ID P14618), C1QC (UniProt ID P02747), CP (UniProt ID P00450), LCP1 (UniProt ID P13796), CAPZB (UniProt ID P47756), ARHGDIB (UniProt ID P52566), and RAB11A (UniProt ID P62491) in the urine sample; and
(c) identifying the subject as having the pulmonary disease or disorder if the one or more proteins have significant differences in abundance, as compared to a control sample.

3. The method of claim 2, wherein the subject is an extremely low gestational age newborn (ELGAN).

4. The method of claim 2, wherein the one or more proteins are chitinase-3-like protein-1 (CHI3L1, also known as YKL-40) and frizzled-6 (FZD6).

5. The method of claim 2, wherein the one or more proteins are matrix metalloproteinase-9 (MMP-9), Tubulin Alpha 3C (TUBA3C), and F-actin-capping protein subunit beta (CAPZB).

6. The method of claim 2, wherein the pulmonary disease is bronchopulmonary dysplasia (BPD).

7. The method of claim 2, wherein the control sample is a urine sample from a healthy subject.

8. The method of claim 7, wherein the healthy subject is an age-matched subject.

9. The method of claim 2, wherein the abundance of the one or more proteins are upregulated in a subject having BPD.

10. The method of claim 9, wherein the one or more proteins are selected from the group consisting of CHI3L1 (UniProt ID P36222), MMP9 (UniProt ID P14780), C2 (UniProt ID P06681), FZD6 (UniProt ID 060353), FKBP1A (UniProt ID P62942), PSMA1 (Uniprot ID P25786), LRRC4B (UniProt ID Q9NT99), C1QC (UniProt ID P02747), CP (UniProt ID P00450), LCP1 (UniProt ID P13796), ARHGDIB (UniProt ID P52566), and RAB11A (UniProt ID P62491).

11. The method of claim 2, further comprising determining that the abundance of one or more proteins is downregulated.

12. The method of claim 11, wherein the one or more proteins are selected from the group consisting of HSPA8 (UniProt ID P111420), HSPA7 (UniProt ID P48741), ACTB (UniProt ID P60709), RPS23 (UniProt ID P62266), CAPN1 (UniProt ID P07384), TUBA3C (UniProt ID PODPH7), PKM (UniProt ID P14618), and CAPZB (UniProt ID P47756).

13. A method of diagnosing bronchopulmonary dysplasia (BPD) in a subject, comprising:
(a) obtaining a urine sample from the subject;
(b) detecting the abundance of one or more proteins selected from the group consisting of CHI3L1 (UniProt ID P36222), MMP9 (UniProt ID P14780), HSPA8 (UniProt ID P111420, C2 (UniProt ID P06681), FZD6 (UniProt ID 060353), HSPA7 (UniProt ID P48741), ACTB (UniProt ID P60709), FKBP1A (UniProt ID P62942), RPS23 (UniProt ID P62266), CAPNI (UniProt ID P07384), PSMA1 (UniProt ID P25786), LRRC4B (UniProt ID Q9NT99), TUBA3C (UniProt ID PODPH7), PKM (UniProt ID P14618), C1QC (UniProt ID P02747), CP (UniProt ID P00450), LCP1 (UniProt ID P13796), CAPZB (UniProt ID P47756), ARHGDIB (UniProt ID P52566), and RAB11A (UniProt ID P62491) in the urine sample;
(c) identifying the subject as bronchopulmonary dysplasia (BPD) if the one or more proteins show significant differences in abundance, as compared to a control sample.

14-15. (canceled)

16. The method of claim 13, wherein the abundance of the one or more proteins are upregulated in a subject having BPD, wherein the one or more proteins are selected from the group consisting of CHI3L1 (UniProt ID P36222), MMP9 (UniProt ID P14780), C2 (UniProt ID P06681), FZD6 (UniProt ID 060353), FKBP1A (UniProt ID P62942), PSMA1 (Uniprot ID P25786), LRRC4B (UniProt ID Q9NT99), C1QC (UniProt ID P02747), CP (UniProt ID P00450), LCP1 (UniProt ID P13796), ARHGDIB (UniProt ID P52566), and RAB11A (UniProt ID P62491).

17. (canceled)

18. The method of claim 13, further comprising determining that the abundance of one or more proteins is downregulated, wherein the one or more proteins are selected from the group consisting of HSPA8 (UniProt ID P111420), HSPA7 (UniProt ID P48741), ACTB (UniProt ID P60709), RPS23 (UniProt ID P62266), CAPN1 (UniProt ID P07384), TUBA3C (UniProt ID PODPH7), PKM (UniProt ID P14618), and CAPZB (UniProt ID P47756).

19. (canceled)

20. The method of claim 13, wherein the one or more proteins are chitinase-3-like protein-1 (CHI3L1, also known as YKL-40) and frizzled-6 (FZD6).

21. The method of claim 13, wherein the one or more proteins are selected from the group consisting of matrix metalloproteinase-9 (MMP-9), Tubulin Alpha 3C (TUBA3C), and F-actin-capping protein subunit beta (CAPZB).

22. A method of predicting a response to a drug for bronchopulmonary dysplasia (BPD) in a subject, comprising:
(a) obtaining a urine sample from the subject;
(b) detecting the abundance of one or more proteins selected from Table 2 in the urine sample; and
(c) identifying the subject as potential responder for the BPD drug if the one or more proteins show significant differences in abundance, compared to a control sample.

23. The method of claim 22, wherein the BPD drug is selected from a small molecule drug, an antibody or antigen-binding fragment thereof, an oligonucleotide or a combination thereof.

24. The method of claim 22, wherein the BPD drug is selected from Marimastat, Captopril, Tacrolimus, Pimecrolimus, Bendroflumethiazide, Celecoxib, Miglitol, Bumetanide, Artenimol, Stiripentol, Artenimol, Dasatinib, Halofuginone, Dalteparin, Artenimol, Cholecystokinin, Oxibendazole, Dasatinib, and Diflucorolone.

25-26. (canceled)

27. The method of claim 13, wherein the subject is an extremely low gestational age newborn (ELGAN).
